(19) United States
(12) Patent Application Publication
(10) Pub. No.: US 2026/0158219 A1
(43) Pub. Date: Jun. 11, 2026

(54) Medicament delivery device with telescopic power spring support

(71) Applicant: SHL Medical AG, Zug (CH)

(72) Inventors: Daniel Carlsson, Nacka Strand (SE); Daniel Säll, Nacka Strand (SE)

(21) Appl. No.: 18/708,021

(22) PCT Filed: Oct. 26, 2022
(86) PCT No.: PCT/EP2022/079913

§ 371 Date: May 7, 2024

(30) Foreign Application Priority Data
Nov. 9, 2021 (EP) 21207283.9

(51) Int. Cl. A61M 5/20; A61M 5/24; A61M 5/315

(52) U.S. Cl. CPC A61M 5/2033; A61M 5/2429; A61M 5/3158; A61M 2005/2411; A61M 2205/581

(57) ABSTRACT

The present disclosure provides a power pack for a medicament delivery device is, including a tubular body having an inner tubular section, and a plunger rod and a power spring arranged within the inner tubular section. An extension sleeve forms a telescopic connection with the inner tubular section and has a connector for connecting the extension sleeve to the power spring, such that during at least part of an expansion of the power spring during activation of the device, the connection sleeve moves with the power spring and provides lateral/radial support for a part of the power spring which (due to the expansion of the power spring) increasingly extends out from a proximal end of the inner tubular section.

### CROSS-REFERENCE TO RELATED APPLICATIONS

The present application is a U.S. National Phase Application pursuant to 35 U.S.C. § 371 of International Application No. PCT/EP2022/079913 filed Oct. 26, 2022, which claims priority to European Patent Application No. 21207283.9 filed Nov. 9, 2021. The entire disclosure contents of these applications are herewith incorporated by reference into the present application.

### TECHNICAL FIELD

The present disclosure relates to the field of medicament delivery devices. In particular, the present disclosure relates to a power pack for such a device having a telescopic power spring support.

### BACKGROUND

Autoinjectors and other types of medicament delivery devices are designed to allow for a user (e.g. a patient) to perform self-administration of a dose of a particular medicament in a controlled way. After positioning the device at the site of the body where the injection is desired, the user activates the device by pushing a release button. Pushing the release button releases a pre-compressed power spring which, when released, in turn drives a plunger rod towards the proximal end of the autoinjector. The plunger rod comes into contact with a syringe in which the medicament to be delivered is stored, and the movement of the plunger rod forces the medicament out of the syringe and into the injection site. In some autoinjectors, the movement of the plunger rod is also responsible for first inserting the needle into the bodily tissue at the injection site.

The power spring can suffer from various buckling phenomena, in which the pre-load of the power spring causes the power spring to bend during its release. Such buckling can lead to a reduced performance and reliability of the injection process and of the medicament delivery device. For optimal performance and reliability, it is therefore desirable that the power spring is radially supported throughout its whole length during expansion.

However, as the power spring can be long compared to other elements of the medicament delivery device, providing radial support over the full possible length of the spring can greatly increase the overall length of the medicament delivery device. This is especially true in situations where the power spring is also responsible for automatically inserting the needle into the injection site, as described above. As it can be desirable to make the medicament delivery devices compact and easier to e.g. store and handle, providing radial support for the power spring in such more compact devices can therefore be problematic.

### SUMMARY

To at least partially solve the above identified problem with providing radial support for the power spring even in compact medicament delivery devices, the present disclosure provides an improved power pack and an improved medicament delivery device, as defined in the accompanying independent claims. Various alternative embodiments are defined in the dependent claims.

According to a first aspect of the present disclosure, a power pack for a medicament delivery device is provided. The power pack includes a tubular body extending along a longitudinal axis. The tubular body includes an inner tubular section arranged fixed relative to the tubular body and also extending along the longitudinal axis. The power pack further includes a plunger rod at least partially arranged within the inner tubular section and movable along the longitudinal axis. The power pack further includes a power spring at least partially arranged within the inner tubular section and expandable along the longitudinal axis and, in a compressed state, at least partially surrounding the plunger rod such that the power spring, when released and expanding along the longitudinal axis during activation of the power pack, drives the plunger rod along the longitudinal axis in a proximal direction of the power pack. The power pack also includes an extension sleeve forming a telescopic connection with the inner tubular section, wherein the extension sleeve is arranged at a proximal end of the inner tubular section and movable along the longitudinal axis in the proximal direction relative to the inner tubular section. The extension sleeve further includes a connector configured for connecting the extension sleeve to the power spring such that, during at least part of the expansion of the power spring, the extension sleeve moves with the power spring in the proximal direction and the extension sleeve thereby provides lateral (or radial) support for at least a part of the power spring extending out from the proximal end of the inner tubular section.

The present disclosure of the power pack according to the first aspect improves upon existing technology in that the telescopic extension sleeve moves together with the spring, and thereby, during the expansion of the power spring, provides a lateral/radial support also for the part of the power spring which, due to the expansion, extends out from and is no longer supported by the inside of the inner tubular section. By providing lateral/radial support over a longer length of the power spring, the risk of a bending/buckling of the power spring is reduced and both the functionality and reliability of the power pack and the medicament delivery device are increased.

In one or more embodiments of the power pack, the inner tubular section may include a guiding track extending along the longitudinal axis. The extension sleeve may include an inner rib protruding laterally/radially inwards from an inside of the extension sleeve, and extending along the longitudinal axis. The telescopic connection may be formed by the rib being received in and guide by the guiding track. When the power spring expands, and the extension sleeve moves with the power spring, a bending/buckling of the part of the power spring which, during the expansion of the power spring, extends out from the inner tubular section can be blocked by the power spring abutting the inner rib.

In one or more embodiments of the power pack, a thickness of the inner rib in a lateral/radial direction may match a thickness of a wall of the inner tubular section. By adjusting the thickness of the inner rib to that of the wall of the inner tubular section, the power spring will not be able to bend/buckle any more than it would if the inner tubular section was instead made to extend further in the proximal direction.

In one or more embodiments of the power pack, the power pack may further include a soft member arranged at a proximal end of the extension sleeve and extending in the proximal direction. If the extension sleeve is movable long enough (with the power spring, and plunger rod) in the proximal direction to hit e.g. a syringe, the soft member may reduce the impact thus created.

In one or more embodiments of the power pack, the connector may be a flexible arm extending laterally inwards such that, during the at least a part of the expansion of the power spring, a (distal) surface of the flexible arm (at least temporary) abuts at least one coil section of the power spring such that the flexible arm moves with the power spring in the proximal direction, thereby causing also said moving of the extension sleeve with the power spring in the proximal direction.

In one or more embodiments of the power pack, the flexible arm is made from sheet metal, other metals or plastics or similar.

In one or more embodiments of the power pack, a stiffness of the flexible arm may be high enough such that a colliding of the flexible arm and one or more coils of the power spring moving in the proximal direction, and/or a bending and subsequent releasing of the flexible arm caused by said colliding, may generate at least one clicking sound. This may provide an audible feedback to a user of the power pack and medicament delivery device about the current state of the injection process.

In one or more embodiments of the power pack, the power pack may further include a release button. The release button may be at least partially arranged within the tubular body and movable along the longitudinal axis (at least in the proximal direction of the power pack). The release button may be configured to, if pushed in the proximal direction, cause an activation of the power pack by releasing the power spring.

In one or more embodiments of the power pack, the inner tubular section may include a distal flexible arm configured to abut a distal rim of the plunger rod to block the power spring from being released and the plunger rod from moving along the longitudinal axis in the proximal direction. The release button may include a protrusion extending from a distal end of the release button in the proximal direction, and configured to, as a result of pushing the release button along the longitudinal axis in the proximal direction, abut a distal surface of the flexible arm of the inner tubular section and cause a bending of the flexible arm of the inner tubular body laterally (or radially) outwards and away from the longitudinal axis, thereby causing the activation of the power pack by preventing the blocking of the power spring from being released and the plunger rod from moving along the longitudinal axis in the proximal direction.

In one or more embodiments of the power pack, during the expansion of the power spring, a proximal end of the power spring may abut a proximal rim of the plunger rod, thus driving the plunger rod along the longitudinal axis in the proximal direction.

According to a second aspect of the present disclosure, a medicament delivery device is provided. The medicament delivery device includes a power pack according to e.g. the first aspect.

Other objects and advantages of the present disclosure will be apparent from the following detailed description, the drawings and the claims. Within the scope of the present disclosure, it is envisaged that all features and advantages described with reference to e.g. power pack of the first aspect are relevant for, apply to, and may be used in combination with also any feature and advantage described with reference to the medicament delivery device of the second aspect, and vice versa.

### BRIEF DESCRIPTION OF THE DRAWINGS

Exemplifying embodiments will now be described below with reference to the accompanying drawings, in which:

FIG. 1A schematically illustrates a perspective view of an embodiment of a medicament delivery device according to the present disclosure;

FIGS. 1B and 1C schematically illustrate cross-sections of an embodiment of a power pack according to the present disclosure;

FIGS. 1D and 1E schematically illustrates perspective views of an embodiment of an extension sleeve according to the present disclosure, and

FIG. 1F schematically illustrates a perspective sectional view of part of an embodiment of a tubular body according to the present disclosure.

In the drawings, like reference numerals will be used for like elements unless stated otherwise. Unless explicitly stated to the contrary, the drawings show only such elements that are necessary to illustrate the example embodiments, while other elements, in the interest of clarity, may be omitted or merely suggested. As illustrated in the Figures, the (absolute or relative) sizes of elements and regions may be exaggerated or understated vis-à-vis their true values for illustrative purposes and, thus, are provided to illustrate the general structures of the embodiments.

### DETAILED DESCRIPTION

Exemplifying embodiments of a medicament delivery device, a power pack, an extension sleeve, and a tubular body according the present disclosure will now be described more fully hereinafter with reference to the accompanying drawings. The drawings show currently preferred embodiments, but the invention may, however, be embodied in many different forms and should not be construed as limited to the embodiments set forth herein; rather, these embodiments are provided for thoroughness and completeness, and fully convey the scope of the present disclosure to the skilled person.

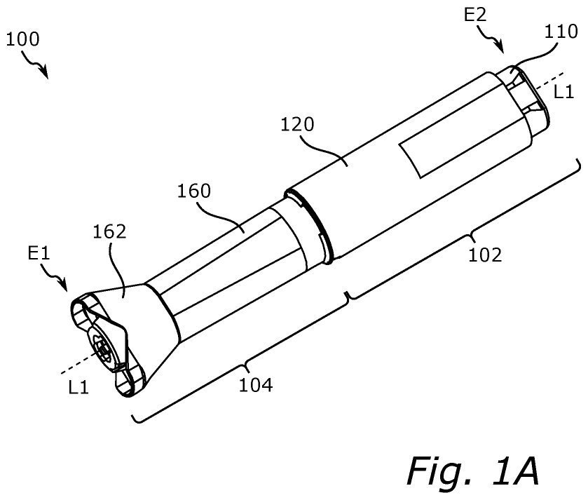

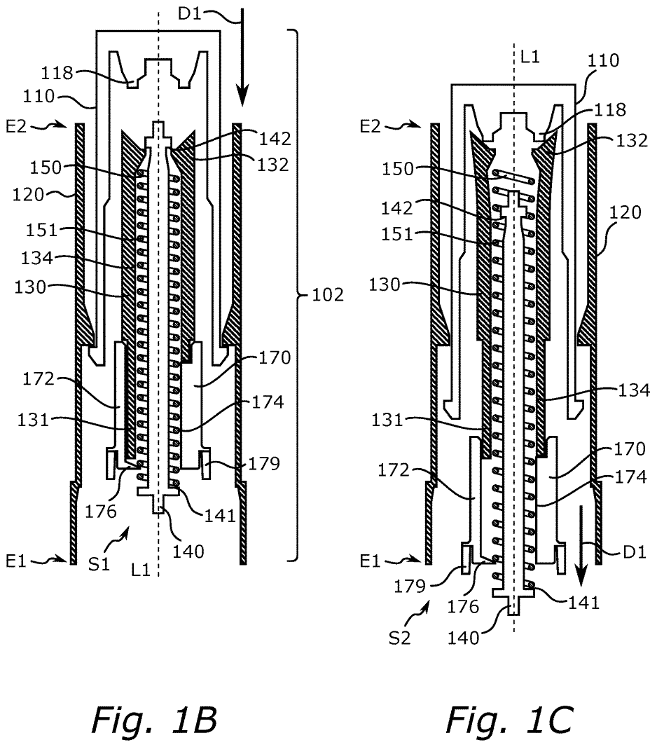

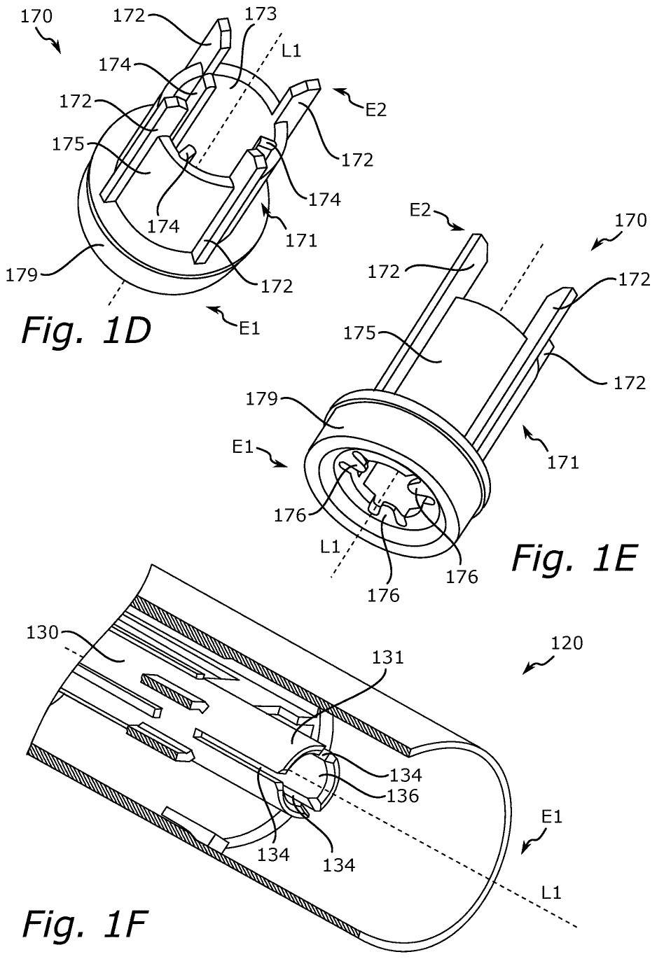

FIG. 1A schematically illustrates a medicament delivery device 100 according to one embodiment of the present disclosure. Hereinafter, the terms “medicament delivery device” and “device” will be used interchangeably.

The device 100 can for example be manufactured as two sub-assemblies, namely a so-called power pack 102 and a so-called front assembly 104, which are joined together during final assembly of the device 100. In other embodiments, it is envisaged that the device 100 may arrive fully assembled from the factory, with the power pack 102 and the front assembly 104 already joined together as illustrated in FIG. 1A. The power pack 102 and the front assembly 104 are arranged such that they both extend along a longitudinal axis L1, from a proximal end E1 to a distal end E2. The power pack 102 includes a tubular body 120 and a release button 110, both extending along the longitudinal axis L1. The release button 110 is at least partially arranged within the tubular body 120 and is movable along the longitudinal axis L1. Here, “movable” implies that the release button can, if not blocked by any other component of the power pack, be pushed at least in a proximal direction D1 relative to the tubular body 120. Such pushing of the release button 110 can be achieved e.g. by a user using a finger, such as a thumb, to apply a force on the release button in the proximal direction.

Although not explicitly indicated in FIG. 1A, it is to be understood that each one of the power pack 102 and the front assembly 104 can also be considered as having its own proximal and distal ends, and that each one of the power pack 102 and the front assembly 104 can also be considered as extending along its own longitudinal axis. When arranged as in FIG. 1A, the longitudinal axis of each sub-assembly aligns with the longitudinal axis L1 of the device as a whole. Likewise, the distal end of the power pack is also the distal end E2 of the device 100, while the proximal end of the front assembly 104 is the proximal end E1 of the device 100. The proximal end of the power pack 102 can be understood as the end of the power pack 102 being closest to the front assembly 104 and, similarly, the distal end of the front assembly 104 can be understood as the end of the front assembly 104 being closest to the release button 110. Similarly, each particular component of the power pack 102 and the front assembly 104 can be defined as having its own proximal and distal ends, and also its own longitudinal axis. When so doing, it can be assumed that the components are all arranged as indicated in FIG. 1A. The references L1, E1 and E2 will be used throughout the present disclosure to indicate such longitudinal axis, proximal end distal ends of each component, as needed. Even if the device 100 is fully assembled, it is envisaged that the various proximal and distal ends of the various components are still as they were before the device 100 was fully assembled, as illustrated in FIG. 1A. The device 100 can be fully assembled by e.g. pushing the distal end of the front assembly 104 into the proximal end of the power pack 102.

The front assembly 104 can optionally include a protective cap 162, which functions e.g. to protect a needle (not shown) of the front assembly 104 from being accessible by a user of the device 100. When the device 100 is to be used, the protective cap 162 can first be removed by the user. In some embodiments, the device 100 can be configured (using e.g. a rotator, also not shown) such that even with the protective cap 162 removed, the device 100 must first be pressed against the injection site before the release button 110 can be used to activate the power pack and the device, and cause the injection of the medicament into the body of the user.

The various components of the device 100 related to the power pack 102, as envisaged by the present disclosure, will now be described in more detail with reference to FIGS. 1B-1F.

FIG. 1B schematically illustrates a cross-section of an embodiment of the power pack 102, where the power pack 102 is not yet assembled with the front assembly. The power pack includes the tubular body 120. The tubular body 120 extends along the longitudinal axis L1 from a proximal end E1 to a distal end E2. The tubular body 120 further includes an inner tubular section 130 which is arranged fixed relative to the tubular body 120 and which also extends along the longitudinal axis L1. It is envisaged that, to fix the inner tubular section 130 with respect to the tubular body 120, one or more support structures (not shown) may for example extend from an inside wall of the tubular body 120 to the inner tubular section 130.

The power pack 102 further includes a plunger rod 140 and a power spring 150, which are both at least partially arranged within the inner tubular section 130. Here, “at least partially” is to be understood that there may be some parts of the power spring 150 and the plunger rod 140 that extends out of the inner tubular section 130, e.g. either at one or both of a proximal end 131 and a distal end of the inner tubular section 130. The power spring 150 includes a plurality of coil sections 151. Each coil section 151 may for example be considered as one complete turn of wire coiled to create the power spring 150. The power spring 150 is arranged such that it can, if not intentionally blocked by any other components of the power spring, expand along the longitudinal axis L1. In the particular embodiment illustrated in FIG. 1B, the power spring 150 is in particular arranged to expand towards the proximal end E1 while it can have no or little expansion in the opposite direction, towards the distal end E2.

In FIG. 1B, the power spring 150 is in a compressed state S1. Here, “compressed” does not necessarily mean fully compressed, but at least compressed enough such that the power spring 150 will expand as described above when the compression is released. In the compressed state S1, the power spring 150 at least partially surrounds the plunger rod 140 such that the power spring 150, when released and expanding (as will be described later herein with reference to FIG. 1C) along the longitudinal axis L1, can drive the plunger rod 140 along the longitudinal axis L1 towards the proximal end E1. In the particular embodiment illustrated in FIG. 1B, this is achieved by a proximal end of the power spring 150 abutting a proximal rim 141 of the plunger rod, while a distal end of the power spring 150 abuts e.g. a distal section of the inner tubular section 130.

In particular, the power pack 102 as envisaged herein also includes an extension sleeve 170 which is arranged at the proximal end 131 of the inner tubular section 130. The extension sleeve 170 is movable along the longitudinal axis L1 in a proximal direction D1 of the power pack 102 and relative to the inner tubular section 130. Thus, the extension sleeve 170 forms a telescopic connection with the inner tubular section 130, as the extension sleeve 170 may be e.g. moved in the distal direction D1 and thereby, as it is connected with the inner tubular section 130, make the inner tubular section 130 appear longer in the proximal direction D1.

More in particular, the extension sleeve 170 includes a connector 176 which is configured to connect the extension sleeve 170 to the power spring 150. As a consequence, if the power spring 150 is expanded (as will be described herein in more detail with reference to FIG. 1C), the extension sleeve 170 can move together with the power spring 150 in the proximal direction D1 during at least part of the expansion of the power spring 150. This can allow the extension sleeve 170 to provide lateral (or radial) support also for at least a part of the power spring 150 which (due to the expansion of the power spring 150) more and more/increasingly extends out from the proximal end 131 of the inner tubular section 130.

One example of how to create the telescopic connection between the extension sleeve 170 and the inner tubular section 130 is also illustrated in FIG. 1B, wherein the extension sleeve 170 is provided with at least one inner rib 174 which is guided and received in a corresponding guiding track of the inner tubular section 130 (as will be described in more detail herein e.g. with reference to FIG. 1F). To provide additional support and stability, the extension sleeve 170 can also be provided with one or more outer ribs 172 which abut an outer surface of the inner tubular section 130, especially when the extension sleeve 170 has moved sufficiently far relative to the inner tubular section 130 in the proximal direction D1. In other embodiments, such outer ribs 172 may be optional, and it is also envisaged that other mechanisms of providing the telescopic connection between the extension sleeve 170 and the inner tubular section 130 are possible, including or not including e.g. the use of the inner rib 174 and corresponding guiding track of the inner tubular section 130.

In the particular example illustrated in FIG. 1B, a soft member 179 is arranged at a proximal end of the extension sleeve 170. The advantage of such a soft member 179 will be described later herein e.g. with reference to FIGS. 1D and 1E. It is, however, envisaged that the soft member 179 can be optional and not present in other envisaged embodiments of the power pack 102.

The power pack 102 can also include a release button 110. The release button 110 is at least partially arranged within the tubular body 120 and is movable (with respect to the tubular body 120) along the longitudinal direction L1 of the tubular body 120. In the position of the various components shown in FIG. 1B, the release button 110 does not yet interact with any other components of a release mechanism for the power spring 150 and plunger rod 140.

Such a release mechanism preferably provides two things. First, it should be able to hold/lock the power spring 150 compressed and the plunger rod 140 still before the release button 110 is pushed. Second, it should be able to release such a hold of the power spring 150 and plunger rod 140 once the release button 110 is pushed, such that the power pack 102 is then activated. One envisaged way of holding/locking the plunger rod 140 and the power spring 150 before the activation of the power pack 102 is illustrated in FIG. 1B and described as follows. A distal end of the power spring 150 abuts e.g. part of the inner tubular section 130, while a proximal end of the power spring 150 abuts a proximal rim 141 of the plunger rod 140. With the release button 110 in the position as shown in FIG. 1B, the power spring 150 is at least partially compressed and exerts a force on the plunger rod 140 in the proximal direction D1 by the proximal end of the power spring 150 pressing against the proximal rim 141 of the plunger rod 140. To keep the plunger rod 140 from being propelled/driven by the power spring 150 in the proximal direction D1, a distal end of the inner tubular section 130 further includes a (distal) flexible arm 132. As shown in FIG. 1B, part of the flexible arm 132 abuts a corresponding distal rim 142 of the plunger rod 140, which blocks the plunger rod 140 from moving in the distal direction D2. As will be described in more detail later with reference to FIG. 1C, the holding/blocking of the plunger rod 140 caused by the flexible arm 132 and the distal rim 142 can be released by pressing of the release button 110 in the proximal direction D1.

In all the figures herein illustrating cross-sections, it should be noted that although some features are shown in a same cross-sectional plane, that must not necessarily be the case. For example, in the power pack 102 illustrated in FIG. 1B, it may for example be envisaged that the features related to the activation/locking of the plunger rod 140 and power spring 150, including e.g. the flexible arm 132 of the inner tubular section 130, are instead arranged such that they would only show up in another cross-sectional plane, e.g. a cross-sectional plane perpendicular or otherwise angled with respect to the cross-sectional plane shown in FIG. 1B. However, for illustrative purposes only, the components described are assumed to be arranged such that a single cross-section can illustrate all described features.

FIG. 1C schematically illustrates a cross-section of the same power pack 102 as in FIG. 1B, but in another state S2 wherein the release and expansion of the power spring 150 towards the proximal end E1 (i.e. in the proximal direction D1) has started.

With the release button 110 pushed along the longitudinal axis L1 towards the proximal end E1, as shown in FIG. 1C, the release button 110 causes an activation of the power pack 102 as follows. At a distal end of an inside of the release button 110, there is provided a protrusion 118 which extends from the distal end of the release button 110 towards the proximal end E1 of the tubular body 120. When the release button 110 is pushed such as shown in FIG. 1C, the protrusion 118 abuts a distal surface of the flexible arm 132 of the inner tubular section 130, and causes a bending of the flexible arm 132 radially outwards. When the flexible arm 132 is bent radially outwards, it is separated from the distal rim 142 of the plunger rod 140 and can no longer prevent/block the plunger rod 140 from being propelled/driven in the proximal direction D1 by the proximal force applied on the proximal rim 141 of the plunger rod 140 by the at least partially compressed power spring 150. As the power spring 150 expands, the plunger rod 140 is accelerated and moved along the longitudinal axis L1 in the proximal direction D1. This completes the activation of the power pack. The plunger rod 140 may then, in turn, reach and interact with e.g. a syringe/ampulla located in the front assembly, and cause an expulsion of a medicament from the syringe/ampulla. The movement of the plunger rod 140 may also, in some embodiments, be responsible for first causing a movement of a needle of the front assembly in the proximal direction, such that the needle is automatically inserted into the delivery site of the user before the medicament is expelled from the syringe/ampulla.

It should be noted that the features related to the activation of the power pack 102, i.e. the holding and release of the power spring 150 and plunger rod 140 (e.g. the release button 110, the protrusion 118, the flexible arm 132 of inner tubular section 130, the plunger rod 140 and its various rims 141 and 142,, etc.) and their interactions are here discussed only for reasons of completeness, even though their exact function, arrangement, configuration and similar is not directly relevant to the core of the present disclosure, which is the provisioning of extension sleeve 170 at the proximal end 131 of the inner tubular section 130.

In particular, and as already touched upon earlier herein and as illustrated in FIG. 1B, simultaneously with activating the power pack 102 by pressing on the release button 110, the connector 176 is configured to mechanically interact with a coil section 151 of the power spring 150 such that, when e.g. the proximal end of the power spring 150 moves in the proximal direction D1, the extension sleeve 170 will also move in the proximal direction D1 relative to the inner tubular section 130. By so doing, lateral/radial support for the power spring 150 is provided over a longer length of the power spring 150 than without the extension sleeve 170. For example, instead of bending/buckling in the state S2, the power spring 150 may abut an inner surface of the extension sleeve 170 (such as e.g. the inner rib 174). The movement of the extension sleeve 170 relative to the inner tubular section 130 can, as described earlier herein, be guided by the inner rib 174 being received in, and guided by, a corresponding guide track 134 of the inner tubular section 130.

Additionally, at least in some embodiments of the power pack 102, it is envisaged that in the proximal direction D1, the possible envisaged travel of e.g. the power spring 150 and plunger rod 140 can be longer than the possible travel of the extension sleeve 170. It may for example be such that the proximal end of the extension sleeve 170 hits part of the syringe before the plunger rod 140 has fully ejected all medicament from the syringe. In order to allow the plunger rod 140 and the power spring 150 to travel further in the proximal direction D1 than the extension sleeve 170, the connector 176 may for example be provided as a flexible arm. Consequently, once the extension sleeve 170 hits e.g. the syringe, continued expansion of the power spring 150 and continued movement of the plunger rod 140 in the distal direction D1 can be allowed by the coil sections 151 of the power spring 150 bending the flexible arm 176, such that they move past the flexible arm 176 on their way further in the proximal direction D1. To reduce the impact of the extension sleeve 170 hitting e.g. the syringe, the soft member 179 can be utilized for this purpose, if included.

The flexible arm 176 may for example be made of sheet metal, preferably from steel. It is however envisaged that other flexible materials can also be used, such as various metals and plastics, and similar.

The material of the flexible arm 176 can be selected such that if the extension sleeve 170 comes to a halt (by e.g. moving far enough in the proximal direction D1 to e.g. hit the syringe, or by any other means which may restrict the maximally allowed movement of the extension sleeve 170 in the proximal direction D1), the flexible arm 176 will bend such that the particular coil section 151 which was earlier responsible for propelling the extension sleeve 170 in the proximal direction D1 can then move past the flexible arm 176. The remaining coil sections 151 of the power spring 150 that also need to move past the flexible arm 176 before the movement of the plunger rod 140 is completed can also do so, due to the flexibility of the flexible arm 176. It can, for example, also be envisaged that it is not a single coil section 151 which is responsible for propelling the extension sleeve 170 all the way until the extension sleeve 170 e.g. hits the syringe, but that the flexible arm 176 is bent easily enough such that coil sections 151 which collide with the flexible arm 176 cause the flexible arm 176 to be (e.g. successively) bent and subsequently released. By so doing, multiple coil sections 151 may be responsible for propelling the extension sleeve 170 until it finally hits e.g. the syringe. It is envisaged that, in such a situation, the propelling of the extension sleeve 170 in the proximal direction D1 can be achieved by each coil section 151 that hits the flexible arm 176 transfers at least some momentum (due to the impact) to the flexible arm 176 in the proximal direction D1.

If the stiffness of the flexible arm 176 is high enough, the above-described colliding of the flexible arm and some of the coil sections 151, and/or the resulting bending and subsequent release of the flexible arm 176 itself, may generate at least one clicking sound (e.g., one clicking sound for each coil section 151 colliding with the flexible arm 176, and/or for each bending and subsequent release of the flexible arm 176). It is assumed that the generated clicking sound(s) can be audible to the user, assuming of course that the hearing capability of the user is not temporarily or permanently limited due to e.g. one or more medical conditions. The generated clicking sound(s) can provide an audible feedback to the user that e.g. the plunger rod 140 has now reached the syringe, and that the injection of the medicament from the device 100 has started. This can help to remove uncertainty for the user regarding in what stage of the injection process the device 100 is currently in.

FIGS. 1D and 1E schematically illustrate various perspective views of an embodiment of the extension sleeve 170. The extension sleeve 170 extends along the longitudinal axis L1 from a proximal end E1 to a distal end E2. A body 171 of the extension sleeve 170 has an inner side 173 and an outer side 175. On the inner side 173 of the body 171, there is provided at least one inner rib 174, which extends along the longitudinal axis L1 and protrudes out from the inner side 173 radially inwards towards the longitudinal axis L1. The inner rib 174 is configured such that it can be received and guided by a corresponding guiding track arranged at the proximal end 131 of the inner tubular section 130 of the tubular body 120. In the particular example illustrated in FIGS. 1D and 1E, there is provided three such inner ribs 174, but it is envisaged that also fewer than three, or more than three such inner ribs 174 may be provided instead. In particular, the exact number of inner ribs 174 may be selected in accordance with e.g. the available number of corresponding guiding tracks of the inner tubular section 130, the desired lateral/radial support for the power spring 150, and similar.

It may for example be envisaged that in some embodiments of the extension sleeve 170, the thickness of the inner rib 174 in the laterally/radially inwards direction is selected such that it matches a thickness of the wall of the inner tubular section 130 in which the corresponding guiding track is provided. If also adjusting the body 171 of the extension sleeve 170 such that its inner diameter matches the outer diameter of the proximal end 131 of the inner tubular section 130, the inner rib 174 will not protrude into the inside of the inner tubular section 130, and will not risk e.g. causing one or more coil sections 151 of the power spring 150 to accidentally get stuck against the inner rib 174. Instead, the inner surface (or side) 136 of the inner tubular section 130 will still be smooth, as the inner rib 174 is flush with this inner surface 136.

Likewise, once the extension sleeve 170 starts to move in the proximal direction D1 relative to the inner tubular section 130, the inner rib 174 will provide a partial continuation of the inner surface 136 of the inner tubular section 130 in the proximal direction D1, and the power spring 150 can be radially/laterally supported as it can abut the inner rib 174 instead of bending or buckling.

In particular, it may be envisaged that an increased number of inner ribs 174 can provide an increased lateral/radial support for the power spring 150. However, as more guiding tracks on the inner tubular section 130 will also be required, a good compromise between the increased lateral/radial support and the reduced rigidity of the proximal end 131 of the inner tubular section 130 (due to the increasing number of guiding tracks) can be for example two, three or four inner ribs in total. Other configurations are of course also possible.

In the particular example illustrated in FIGS. 1D and 1E, the extension sleeve 170 is further provided with at least one outer rib 172. The outer rib 172 also extends along the longitudinal direction and protrudes laterally (or radially) outwards from the outer side 175 of the body 171 and away from the longitudinal axis L1. The outer rib 172 may for example provide additional stability for the extension sleeve 170 when moving relative to the inner tubular section 130.

As can be seen in FIG. 1E, the extension sleeve 170 has, at its proximal end E1, the connector in form of at least one flexible arm 176. The flexible arm 176 extends laterally/radially inwards towards the longitudinal axis L1 (from e.g. the inner side 173 of the body 171). The extension in this direction is long enough for the flexible arm 176 to come into contact with the power spring 150, when the extension sleeve 170 is mounted to the inner tubular section 130 of the power pack 102. The exact length of this extension may depend e.g. on the diameter of the power spring 150, the inner diameter of the extension sleeve 170, the material of the flexible arm 176, etc. In the particular example illustrated in FIG. 1E, there is provided three such flexible arms 176. It is however envisaged also that there may be only a single flexible arm 176, two flexible arms 176, or even more than three flexible arms 176.

As can also be seen in FIG. 1E, there is also provided the soft member 179 at the proximal end E1 of the extension sleeve 170. As discussed earlier herein, the soft member 179 may for example help to dampen an impact of the extension sleeve 170 hitting e.g. a syringe in the front assembly 104 of the medicament delivery device 100. The soft member 179 may for example be made of a soft material such as rubber, or in particular of a thermoplastic elastomer (TPE), or similar. However, it is envisaged that the soft member 179 is optional and that it may not be included in other envisaged embodiments of the power pack 102 and extension sleeve 170.

By using the flexibility of the flexible arm 176, the extension sleeve 170 can be mounted to the inner tubular section 130 even after the power spring 150 and plunger rod 140 have already been inserted into the inner tubular section 130.

FIG. 1F schematically illustrates a perspective sectional view of an embodiment of the tubular body 120. FIG. 1F does not show all of the tubular body 130, but only the parts towards its proximal end E1, and in particular the proximal end 131 of the inner tubular section 130.

The inner tubular section 130 includes, at its proximal end 131, at least one guiding track 134. The guiding track 134 extends along the longitudinal axis L1. In the particular example illustrated in FIG. 1F, the guiding track 134 starts at the very edge of the proximal end 131 of the inner tubular section 130, and extends towards the distal end of the inner tubular section 130. The guiding track 134 goes all the way through the wall of the inner tubular section 130, from the outside of the proximal end 131 of the inner tubular section 130 to an inner side 136 of the inner tubular section 130.

The size and length of extension of the guiding track 134 is configured such that it matches the corresponding inner rib 174 of the extension sleeve 170, such that this inner rib 174 can be received and guided in the guiding track 134 once the extension sleeve 170 is mounted on the outside of the proximal end 131 of the inner tubular section 130. In the particular example illustrated in FIG. 1F, there is provided three guiding tracks 134, matching the three inner ribs 174 of the extension sleeve 170 illustrated in FIGS. 1D and 1E. Of course, it is envisaged that there may be a different number of guiding tracks 134 than exactly three, and that the exact number of guiding tacks 134 may be adapted to e.g. the exact number of inner ribs 174 of the extension sleeve 170.

In summary of FIGS. 1A-1F and the envisaged solution presented herein, it is concluded that the various components of the device 100, and in particular of the power pack 102 (such as the extension sleeve 170), are configured to interact such that the extension sleeve 170 forms a telescopic connection with the inner tubular section 130 and is configured to move with the power spring 150 during at least part of the expansion of the power spring 150, such that it then provides an effective extension of at least part of the inner side/surface 136 of the inner tubular section 130. This helps to provide an improved lateral/radial support of the power spring 150 over a longer part of the total length of the power spring, especially during the expansion. This in turn helps to prevent the power spring 150 from buckling or bending when expanding, as the power spring 150 can, instead of bending or buckling, abut the inside (e.g. the inner rib 174) of the extension sleeve 170. This provides an improved functionality and reliability of the power pack 102 and the device 100. At the same time, as the extension sleeve is telescopic, the overall length of the power pack 102 and the device 100 does not need to be increased, and the power pack 102 and the device 100 can still be manufactured compact and easy to handle/store for the user.

Although not explicitly disclosed in any drawings, or at least not explicitly further described in the description so far, it is herein envisaged that the medicament delivery device may also include additional components, including e.g. a delivery member, the protective cap for the delivery member, an ampulla for storing the dose of medicament, one or more guiding rods and or other structures for the arrangement of a delivery member cover, a rotator for blocking pushing of the release button before e.g. the delivery member cover is pressed against the injection site, etc. It is envisaged that any such components are not directly relevant for describing the core idea underlying the present disclosure (i.e. the provisioning of improved radial/lateral support for the power spring during its expansion), and any further illustration and/or discussion of such additional components has therefore, for reasons of clarity, been left out of the present disclosure.

Medicament delivery devices as envisaged herein may for example be autoinjectors containing insulin, allergy medicaments, epinephrine, migraine medicaments, atropine, or any other medicament/drug for which the use of a medicament delivery device as described herein is suitable. The device can e.g. be of a single-use type (i.e. disposable). Delivery members may include needles, parts of syringes, syrettes, or other suitable structures.

In the present disclosure, the term “longitudinal axis” of the device refers to an axis extending from a proximal end of the device to a distal end of the device, typically a central axis along the device in the direction of longest extension of the device. The term “distal end” refers to the part/end of the device, or the parts/ends of the members thereof, which under use of the device is/are located furthest away from the dose delivery/injection site. Correspondingly, the term “proximal end” refers to the part/end of the device, or the parts/ends of the members thereof, which under use of the device is/are located closest to the dose delivery/injection site. As described further above, this also apply to the various components of the device itself, such as the power pack, the front assembly, the release button, the tubular body of the power pack, the extension sleeve, etc., which can all be considered as each having their own longitudinal axis (where applicable), and which, when assembled in the device, can all be considered as having also their own corresponding proximal and distal ends.

Although features and elements may be described above in particular combinations, each feature or element may be used alone without the other features and elements or in various combinations with or without other features and elements. Additionally, variations to the disclosed embodiments may be understood and effected by the skilled person in practicing the claimed invention, from a study of the drawings, the disclosure, and the appended claims.

In the claims, the words “comprising” and “including” does not exclude other elements, and the indefinite article “a” or “an” does not exclude a plurality. The mere fact that certain features are recited in mutually different dependent claims does not indicate that a combination of these features cannot be used to advantage.

1-11. (canceled)

12. A power pack for a medicament delivery device, comprising:
a tubular body extending along a longitudinal axis, the tubular body including an inner tubular section arranged fixed relative to the tubular body and also extending along the longitudinal axis;
a plunger rod at least partially arranged within the inner tubular section and movable along the longitudinal axis;
a power spring at least partially arranged within the inner tubular section and expandable along the longitudinal axis and, in a compressed state, at least partially surrounding the plunger rod such that the power spring, when released and expanding along the longitudinal axis during activation of the power pack, drives the plunger rod along the longitudinal axis in a proximal direction of the power pack, and
an extension sleeve forming a telescopic connection with the inner tubular section, wherein the extension sleeve is arranged at a proximal end of the inner tubular section and movable along the longitudinal axis in the proximal direction relative to the inner tubular section,
wherein the extension sleeve further includes a connector configured for connecting the extension sleeve to the power spring such that, during at least part of the expansion of the power spring, the extension sleeve moves with the power spring in the proximal direction and the extension sleeve thereby provides lateral support for at least a part of the power spring extending out from the proximal end of the inner tubular section.

13. The power pack according to claim 12, wherein the inner tubular section includes a guiding track extending along the longitudinal axis, the extension sleeve includes an inner rib protruding laterally inwards from an inner side of the extension sleeve and extending along the longitudinal axis, and wherein the telescopic connection is formed by the rib being received in and guided by the guiding track.

14. The power pack according to claim 13, wherein a thickness of the inner rib in a lateral direction matches a thickness of a wall of the inner tubular section.

15. The power pack according to claim 12, further including a soft member arranged at a proximal end of the extension sleeve and extending in the proximal direction.

16. The power pack according to claim 12, wherein the connector is a flexible arm extending laterally inwards towards the longitudinal axis such that, during at least part of the expansion of the power spring, a surface of the flexible arm abuts at least one coil section of the power spring such that the flexible arm moves with the power spring in the proximal direction, thereby causing also said moving of the extension sleeve with the power spring in the proximal direction.

17. The power pack according to claim 16, wherein the flexible arm is made from sheet metal, preferably from steel.

18. The power pack according to claim 16, wherein a stiffness of the flexible arm is high enough such that a colliding of the flexible arm and one or more coils of the power spring moving in the proximal direction, and/or a bending and subsequent releasing of the flexible arm caused by said colliding, generates at least one clicking sound.

19. The power pack according to claim 12, further comprising:
a release button arranged within the tubular body and movable along the longitudinal axis, and configured to, if pushed along the longitudinal axis in the proximal direction, cause the activation of the power pack by releasing the power spring.

20. The power pack according to claim 19, wherein:
the inner tubular section includes a distal flexible arm configured to abut a distal rim of the plunger rod to block the power spring from being released and the plunger rod from moving along the longitudinal axis in the proximal direction, and
the release button includes a protrusion extending from a distal end of the release button in the proximal direction, and configured to, as a result of pushing the release button along the longitudinal axis in the proximal direction, abut a distal surface of the flexible arm of the inner tubular section and cause a bending of the flexible arm of the inner tubular section laterally outwards and away from the longitudinal axis, thereby causing the activation of the power pack by preventing the blocking of the power spring from being released and the plunger rod from moving along the longitudinal axis in the proximal direction.

21. The power pack according to claim 12, wherein:
during the expansion of the power spring, a proximal end of the power spring abuts a proximal rim of the plunger rod, thus driving the plunger rod along the longitudinal axis in the proximal direction.

22. A medicament delivery device, comprising a power pack according to claim 12.
